(19) United States
(12) Patent Application Publication
(10) Pub. No.: US 2025/0059059 A1
(43) Pub. Date: Feb. 20, 2025

(54) COMPOSITE PARTICLE PRODUCTION METHOD AND COMPOSITE PARTICLE

(71) Applicant: Panasonic Intellectual Property Management Co., Ltd., Osaka (JP)

(72) Inventors: TAKESHI KOIWASAKI, Osaka (JP); HISAO NAGAI, Osaka (JP); HIROKI MARUYAMA, Osaka (JP)

(21) Appl. No.: 18/937,721

(22) Filed: Nov. 5, 2024

Related U.S. Application Data
(63) Continuation of application No. PCT/JP2023/013763, filed on Apr. 3, 2023.

(30) Foreign Application Priority Data
May 17, 2022 (JP) 2022-080762

(51) Int. Cl. C01G 23/047; C08K 3/22

(52) U.S. Cl. CPC C01G 23/047; C08K 3/22; C01P 2004/01; C01P 2004/61; C01P 2004/62; C01P 2004/64; C08K 2003/2241; C08K 2003/2248; C08K 2201/005

(57) ABSTRACT

A method of manufacturing a composite particle includes: a step of preparing a first raw material including an element selected from any of copper, molybdenum, and silver, and a second raw material including one or more types of elements selected from aluminum, titanium, zirconium, hafnium, iron, yttrium, niobium, tantalum, silicon, calcium, magnesium, tungsten, indium, tin, germanium, nickel, zinc, and molybdenum; and a thermal plasma evaporation and cooling step of introducing the prepared first and second raw materials into thermal plasma to evaporate the first raw materials, and cooling the evaporated first raw materials to generate a composite particle. The composite particle includes the second raw material, and a fine particle carried on a surface of the second raw material and generated from the first raw material having an average particle size of 0.5 nm or more and 300 nm or less.

### TECHNICAL FIELD

The present disclosure relates to a method of manufacturing a composite particle in which a large particle and a small particle are compounded, and a composite particle.

### BACKGROUND ART

In recent years, there has been proposed a visible-light-responsive photocatalyst of titanium oxide in which nanometer-order copper compound fine particles exhibiting photocatalytic activity even under indoor light such as a fluorescent lamp are carried on the surface (see, for example, PTL 1).

As a method of manufacturing these copper compound-carried titanium oxides, a manufacturing method by a liquid phase method is known. For example, PTL 1 discloses a manufacturing method in which a reducing agent for reducing divalent copper to monovalent copper is added to a suspension in which titanium oxide having a rutile type titanium oxide content of 50 mol % or more and a divalent copper compound are blended.

Furthermore, as a method of manufacturing a composite particle using a plurality of raw materials, there is a method of manufacturing large particles and small particles, respectively, and attaching the small particles to the surfaces of the large particles using a fluidized bed dry granulation method or a dry mechanical particle compounding method (See, for example, PTL 2.).

### CITATIONS LIST

### Patent Literature

PTL 1: WO 2013/002151 A
PTL 2: Unexamined Japanese Patent Publication No. 2003-275281

### SUMMARY OF THE INVENTION

In PTL 1, titanium oxide having high crystallinity is synthesized by a gas phase method, a divalent copper compound is blended with the titanium oxide, a suspension is stirred and prepared, and thereafter, a reducing agent, such as alkali metal, alkaline earth metal, aluminum, zinc, amalgam of alkali metal or zinc, a hydride of boron or aluminum, a metal salt in a low oxidation state, hydrogen sulfide, sulfide, thiosulfate, oxalic acid, formic acid, ascorbic acid, a substance having an aldehyde bond, and an alcohol compound containing phenol, is further added to reduce divalent copper (Cu(II)) to monovalent copper (Cu(I)).

However, in the conventional manufacturing method, there are a plurality of steps, the manufacturing cost is high, and synthesis in a liquid phase is included, so that usable solvents are limited, and when using the produced particles, complicated treatment such as solvent substitution may be required. Moreover, there is a problem in that it is difficult to adjust the reducing agent and the reducing agent remains as impurities.

In PTL 2, a process of manufacturing large particles and small particles and mixing the large particles and the small particles is provided, the process includes a plurality of steps, and the manufacturing cost increases.

In consideration of the above-described conventional problems, an object of the present disclosure is to provide a method of manufacturing a composite particle capable of efficiently synthesizing composite particles of different materials, and to provide a composite particle having good fluidity and excellent dispersibility.

In order to achieve the above object, a method of manufacturing a composite particle according to the present disclosure includes: a step of preparing a first raw material including an element selected from any of copper, molybdenum, and silver, and a second raw material including one or more types of elements selected from aluminum, titanium, zirconium, hafnium, iron, yttrium, niobium, tantalum, silicon, calcium, magnesium, tungsten, indium, tin, germanium, nickel, zinc, and molybdenum; and a thermal plasma evaporation and cooling step of introducing the prepared first and second raw materials into thermal plasma to evaporate the first raw materials, and cooling the evaporated first raw materials to generate a composite particle. The composite particle includes the second raw material, and a fine particle carried on a surface of the second raw material and generated from the first raw material having an average particle size of 0.5 nm or more and 300 nm or less.

Furthermore, in order to achieve the above object, a composite particle according to the present disclosure includes: a base material particle having an average particle size of more than 0.3 μm and 100 μm or less and including a compound containing an oxide of one type of element selected from aluminum, titanium, zirconium, hafnium, iron, yttrium, niobium, tantalum, silicon, calcium, magnesium, tungsten, indium, tin, germanium, nickel, zinc, and molybdenum; and a fine particle having an average particle size of 0.5 nm or more and 300 nm or less, containing at least one of cuprous oxide, copper oxide, copper, molybdenum oxide, silver, or silver oxide, and being present on a surface of the base material particle.

Moreover, the composite particle according to the present disclosure includes: a base material particle having an average particle size of more than 0.3 μm and 100 μm or less and including a compound containing an oxide of one type of element selected from aluminum, titanium, zirconium, hafnium, iron, yttrium, niobium, tantalum, silicon, calcium, magnesium, tungsten, indium, tin, germanium, nickel, zinc, and molybdenum; and a fine particle having an average particle size of 0.5 nm or more and 300 nm or less, including a compound containing an oxide of one type of element selected from aluminum, titanium, zirconium, hafnium, iron, yttrium, niobium, tantalum, silicon, calcium, magnesium, tungsten, indium, tin, germanium, nickel, and zinc and including at least two of cuprous oxide, copper oxide, copper, molybdenum oxide, silver, or silver oxide, and being present on a surface of the base material particle.

According to the method of manufacturing a composite particle according to the present disclosure, it is possible to easily provide a composite particle of different materials on which a nano-level film or fine particle having excellent dispersibility is formed or attached.

### BRIEF DESCRIPTION OF THE DRAWINGS

FIG. 1 is a diagram illustrating an example of a flow of a method of manufacturing a composite particle according to a first exemplary embodiment.

FIG. 2 is a schematic cross-sectional view illustrating a cross-sectional configuration of a thermal plasma device.

FIG. 3 is a schematic cross-sectional plan view of a thermal plasma device according to the first exemplary embodiment in a state where an electrode portion is cut in a lateral direction.

FIG. 4 is an electron microscope image of a composite raw material in the method of manufacturing a composite particle according to the first exemplary embodiment.

FIG. 5 is an electron microscopic image of a composite particle obtained by the method of manufacturing a composite particle according to the first exemplary embodiment.

### DESCRIPTION OF EMBODIMENT

A method of manufacturing a composite particle according to a first aspect includes: a step of preparing a first raw material including an element selected from any of copper, molybdenum, and silver, and a second raw material including one or more types of elements selected from aluminum, titanium, zirconium, hafnium, iron, yttrium, niobium, tantalum, silicon, calcium, magnesium, tungsten, indium, tin, germanium, nickel, zinc, and molybdenum; and a thermal plasma evaporation and cooling step of introducing each of the prepared raw materials into thermal plasma to evaporate the first raw materials, and cooling the evaporated first raw materials to generate a composite particle in which a fine particle generated from the first raw material having an average particle size of 0.5 nm or more and 300 nm or less is carried on a surface of the second raw material.

In the method of manufacturing a composite particle according to a second aspect, in the above-described first aspect, the thermal plasma evaporation and cooling step may include a step of controlling the thermal plasma not to evaporate more than or equal to 10 wt % of the second raw material.

In the method of manufacturing a composite particle according to a third aspect, in the above-described first or second aspect, the second raw material may have a melting point higher than a melting point of the first raw material.

In the method of manufacturing a composite particle according to a fourth aspect, in any one of the above-described first to third aspects, in the step of preparing the first raw material and the second raw material, the second raw material may be a particle, and the first raw material may be carried or coated on a surface of the particle of the second raw material.

In the method of manufacturing a composite particle according to a fifth aspect, in any one of the above-described first to fourth aspects, the second raw material may be a particle, and the particle of the second raw material may have a spherical shape without a corner that is a pointed portion created by intersection of two surfaces.

In the method of manufacturing a composite particle according to a sixth aspect, in any one of the above-described first to fifth aspects, both the first raw material and the second raw material may be particles, and the first raw material may have a particle size of 0.2 times or less a particle size of the second raw material.

In the method of manufacturing a composite particle according to a seventh aspect, in any one of the above-described first to sixth aspects, the second raw material may be a secondary particle granulated from a primary particle.

In the method of manufacturing a composite particle according to an eighth aspect, in any one of the above-described first to seventh aspects, the thermal plasma evaporation and cooling step includes a step of supplying a cooling gas to a terminal portion of the thermal plasma.

In the method of manufacturing a composite particle according to a ninth aspect, in any one of the above-described first to eighth aspects, the thermal plasma evaporation and cooling step includes a step of controlling the thermal plasma to preferentially evaporate the first raw material by controlling at least one of a temperature of the thermal plasma, a heating time by the thermal plasma, a temperature distribution of the thermal plasma, a gas species of the thermal plasma, a pressure of the thermal plasma, a supply position, a flow rate, and a gas species of a cooling gas of the thermal plasma, a type of a composite raw material in which first raw material 61 is carried or coated on a surface of a particle of second raw material 62, average particle sizes of a primary particle and a secondary particle, a carrying form, and a supply medium of the composite raw material.

A composite particle according to a tenth aspect includes: a base material particle having an average particle size of more than 0.3 μm and 100 μm or less and including a compound containing an oxide of one type of element selected from aluminum, titanium, zirconium, hafnium, iron, yttrium, niobium, tantalum, silicon, calcium, magnesium, tungsten, indium, tin, germanium, nickel, zinc, and molybdenum; and a copper, molybdenum, or silver element-containing particle having an average particle size of 0.5 nm or more and 300 nm or less, containing at least one of cuprous oxide, copper oxide, copper, molybdenum oxide, silver, or silver oxide, and being present on a surface of the base material particle.

A composite particle according to an eleventh aspect includes: a base material particle having an average particle size of more than 0.3 μm and 100 μm or less and including a compound containing an oxide of one type of element selected from aluminum, titanium, zirconium, hafnium, iron, yttrium, niobium, tantalum, silicon, calcium, magnesium, tungsten, indium, tin, germanium, nickel, zinc, and molybdenum; and a fine particle having an average particle size of 0.5 nm or more and 300 nm or less, including a compound containing an oxide of one type of element selected from aluminum, titanium, zirconium, hafnium, iron, yttrium, niobium, tantalum, silicon, calcium, magnesium, tungsten, indium, tin, germanium, nickel, and zinc and including at least two of cuprous oxide, copper oxide, copper, molybdenum oxide, silver, or silver oxide, and being present on a surface of the base material particle.

In the composite particle according to a twelfth aspect, in the above-described tenth or eleventh aspect, the base material particle may have a spherical shape in which there is no corner that is a pointed portion created by intersection of two surfaces, and as shown in Formula (1), a difference between a radius B of a minimum spherical surface inscribed in a surface of the base material particle and a maximum value A of a distance from a center of the minimum spherical surface to the surface of the base material particle is 3/10 or less of the maximum value A.

$$( A - B ) \leq 0.3 \times A \qquad Formula ( 1 )$$

A resin composition according to a thirteenth aspect contains the composite particle according to any one of the above-described tenth to twelfth aspects.

A resin molded body according to a fourteenth aspect contains the composite particle according to any one of the above-described tenth to twelfth aspects.

A metal and ceramic molded body according to a fifteenth aspect contains the composite particle according to any one of the above-described tenth to twelfth aspects.

Hereinafter, a method of manufacturing a composite particle and a composite particle according to an exemplary embodiment will be described in detail with reference to the drawings.

Note that the exemplary embodiment described below are intended to provide comprehensive or specific examples of the present disclosure. Numerical values, shapes, materials, constituent elements, arrangement positions and connection forms of the constituent elements, steps, processing order of the steps, and the like illustrated in the following exemplary embodiment are just an example, and are not intended to limit the present disclosure. Furthermore, those components introduced in the following exemplary embodiment that are not recited in the independent claim(s) representing the most superordinate concept are illustrated herein as optional components. In the drawings, substantially identical configurations are denoted by identical reference numerals, and overlapped descriptions may be omitted or simplified.

Furthermore, various elements illustrated in the drawings are only schematically illustrated for the present disclosure to be understood, and a dimensional ratio, appearance, or the like in the drawings may differ from actual ones.

### First Exemplary Embodiment

### [Method of Manufacturing Composite Particle]

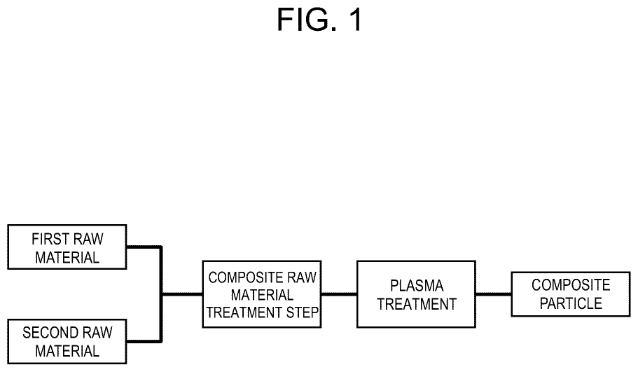

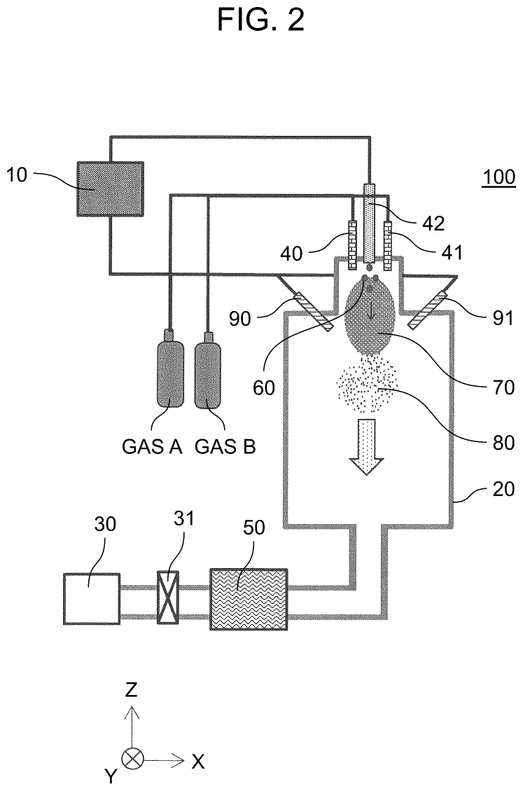

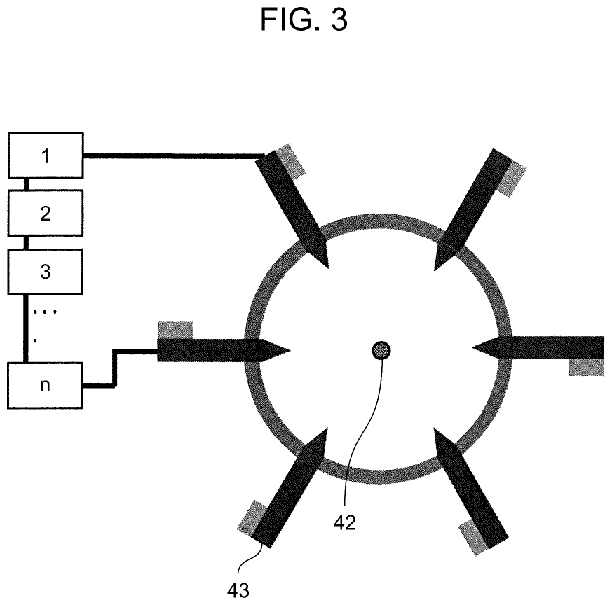

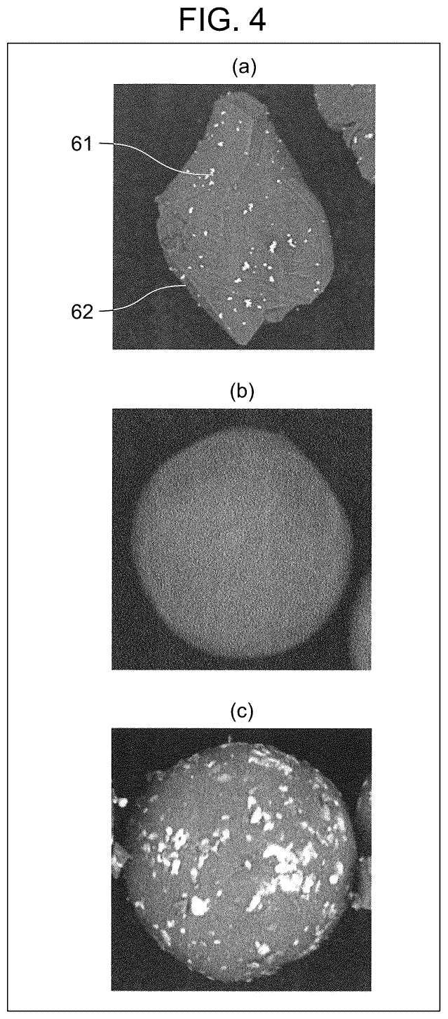

First, a method of manufacturing a composite particle according to the first exemplary embodiment will be described with reference to FIGS. 1 to 3. FIG. 1 is a diagram illustrating an example of a flow of a method of manufacturing composite particle 80 according to the first exemplary embodiment. FIG. 2 is a schematic cross-sectional view illustrating a cross-sectional configuration of thermal plasma device 100. FIG. 3 is a schematic cross-sectional plan view of a thermal plasma device according to the first exemplary embodiment in a state where an electrode portion is cut in a lateral direction. FIG. 4 is an electron microscope image of composite raw material 60 according to the first exemplary embodiment. Note that the entire width of FIG. 4 is about 10 μm. Furthermore, in FIG. 2, for convenience, a vertically upward direction is defined as a Z direction, and a right hand of the paper surface in a horizontal plane is defined as an X direction. A direction from a front side to a back side of the paper surface is a Y direction.

A method of manufacturing composite particles 80 according to the first exemplary embodiment includes a step of preparing first raw material 61 and second raw material 62, and a composite particle generation step of introducing each raw material into thermal plasma 70 (see FIG. 2) and evaporating and cooling first raw material 61 to generate composite particles 80 in which fine particles generated from first raw material 61 are carried on a surface of second raw material 62. That is, composite particles 80 generated include the second raw material 62 and fine particles generated from the first raw material 61 carried on the surface of the second raw material 62. An average particle size of the fine particles generated from first raw material 61 is 0.5 nm or more and 300 nm or less.

In the step of preparing the raw materials, first raw material 61 including one type of element selected from copper and molybdenum and second raw material 62 including one type of element selected from aluminum, titanium, zirconium, hafnium, iron, yttrium, niobium, tantalum, silicon, calcium, magnesium, tungsten, indium, tin, germanium, nickel, zinc, and molybdenum are prepared.

In the composite particle generation step, each prepared raw material is introduced into thermal plasma to evaporate first raw material 61, and evaporated first raw material 61 is cooled. As a result, it is possible to easily manufacture composite particles 80 in which fine particles generated from first raw material 61 including one type of element selected from copper, molybdenum, and silver having an average particle size of 0.5 nm or more and 300 nm or less are carried on the surface of second raw material 62 including one type of element selected from aluminum, titanium, zirconium, hafnium, iron, yttrium, niobium, tantalum, silicon, calcium, magnesium, tungsten, indium, tin, germanium, nickel, zinc, and molybdenum.

According to the method of manufacturing a composite particle, a composite particle excellent in dispersibility can be obtained.

Moreover, in the step of preparing first raw material 61 and second raw material 62, second raw material 62 may be particles, and first raw material 61 may be carried or coated on the surfaces of the particles of second raw material 62 to form composite raw material 60. That is, composite raw material 60 includes second raw material 62 that is particles, and first raw material 61 carried or coated on the surface of second raw material 62. By using such composite raw material 60, it is possible to improve the evaporation efficiency of first raw material 61 in the composite particle generation step and to uniformly carry the first raw material on the surface of second raw material 62.

Moreover, when second raw material 62 is particles and the particles of second raw material 62 have no corner that is a pointed portion created by intersection of two surfaces, in the step of preparing first raw material 61 and second raw material 62, first raw material 61 can be more uniformly carried or thinly coated on the surfaces of the particles of second raw material 62 at the time of forming composite raw material 60, the evaporation efficiency of first raw material 61 in the composite particle generation step can be improved, and first raw material 61 can be uniformly carried on the surfaces of the particles of second raw material 62.

Moreover, it is sufficient that both first raw material 61 and second raw material 62 are particles, and a particle size of first raw material 61 is 0.2 times or less a particle size of second raw material 62. In this case, in the step of preparing first raw material 61 and second raw material 62, first raw material 61 can be more uniformly carried or coated on the surfaces of the particles of second raw material 62 when composite raw material 60 is formed. As a result, the evaporation efficiency of first raw material 61 in the composite particle generation step can be improved, and first raw material 61 can be uniformly carried on the surface of second raw material 62.

Moreover, second raw material 62 may be secondary particles granulated from the primary particles. That is, even particles having a small particle size, which have poor fluidity and are difficult to transport, can be transported by granulating the particles into secondary particles.

Hereinafter, the method of manufacturing composite particle 80 according to the first exemplary embodiment will be described with an example.

As exemplified in FIG. 1, prepared first raw material 61 (here, CuO) and second raw material 62 (here, TiO₂) are used in the method of manufacturing composite particle 80 according to the first exemplary embodiment. The method includes a mixing step (step S1) of mixing first raw material 61 while crushing, pulverizing, carrying or coating the first raw material so as not to pulverize second raw material 62, and a composite particle formation step (step S2) of introducing composite raw material 60 obtained in step S1 into a thermal plasma device, evaporating first raw material 61, cooling evaporated first raw material 61 to be micronized, and carrying the first raw material on a surface of the second raw material 62.

Hereinafter, the method of manufacturing composite particle 80 will be described more specifically. Composite particle 80 is, for example, a composite particle in which Cu₂O particles are carried on surfaces of TiO₂ particles as base material particles.

(a) In the mixing step (step S1), rutile TiO₂ (particles of second raw material 62) having an average particle size of 8 μm as a raw material of TiO₂ and CuO (particles of first raw material 61) having an average particle size of 1 μm as a raw material of Cu₂O are used, and CuO is mixed while being crushed or pulverized so as not to pulverize TiO₂. For example, TiO₂ and CuO are prepared at a weight ratio of 99.5:0.5, and mixed in a mortar. As a result, composite raw material 60 is obtained.

Furthermore, as composite raw material 60 to be introduced into the thermal plasma, a composite raw material in which first raw material 61 to be evaporated is carried or coated on the surface of second raw material 62 as shown in part (a) of FIG. 4 is used. As a result, first raw material 61 on the surface can be preferentially evaporated, and the fine particles generated from first raw material 61 are uniformly carried on the surface of second raw material 62, so that composite particles 80 in which the fine particles having a small variation in particle size are uniformly carried can be manufactured.

Furthermore, in composite raw material 60 to be introduced into the thermal plasma, as illustrated in part (b) of FIG. 4, second raw material 62 is particles, and the particles of second raw material 62 need not have a corner that is a pointed portion created by intersection of two surfaces. This makes it possible to obtain composite raw material 60 in which first raw material 61 is more uniformly carried or coated on the surface of second raw material 62 as illustrated in part (c) of FIG. 4. Furthermore, since the corner disappears, second raw material 62 is less likely to evaporate, and the evaporation of second raw material 62 can be suppressed. In the present exemplary embodiment, first raw material 61 is only carried and partially crushed and formed into a film and coated on the surface of second raw material 62. Moreover, by prolonging the treatment time or treating the particles by an optimum force or method, the particles can be further crushed to increase an area covered with the film. Furthermore, a coating area can also be increased by a mixing ratio of first raw material 61 and second raw material 62. In addition, coating can be performed by a method such as coating using a solution and a gas, vapor deposition, or sputtering, and also, coating of a thin film can be performed. By subjecting obtained composite raw material 60 to thermal plasma treatment, it is possible to manufacture composite particles 80 in which the fine particles generated from first raw material 61 having a small variation in particle size are uniformly carried on the surface of second raw material 62.

Furthermore, in composite raw material 60 to be introduced into the thermal plasma, first raw material 61 and second raw material 62 may be both particles, and the particle size of first raw material 61 may be 0.2 times or less the particle size of second raw material 62. This makes it possible to obtain composite raw material 60 in which first raw material 61 is more uniformly carried or coated on the surface of second raw material 62. Furthermore, in a case where a thickness of second raw material 62 is 0.3 μm or less, it is easy to evaporate, and when the thickness is larger than 0.3 μm, it is possible to suppress evaporation of second raw material 62. By subjecting obtained composite raw material 60 to thermal plasma treatment, it is possible to manufacture composite particles 80 in which the fine particles generated from first raw material 61 having a small variation in particle size are uniformly carried on the surface of second raw material 62.

Furthermore, since the surface area increases as the particle size decreases, the fluidity of the particles deteriorates, and it becomes difficult to transport the particles in a dry manner. However, in composite raw material 60 to be introduced into the thermal plasma, second raw material 62 may be secondary particles granulated from primary particles with, for example, an organic binder or the like. In this case, the particles are secondary particles having a large particle size until being introduced into the thermal plasma, have good fluidity, and can be transported in a dry manner. Thereafter, after the introduction of the thermal plasma, the binder as an organic substance is evaporated, the secondary particles are decomposed into primary particles, and fine particles generated from first raw material 61 are carried on the surfaces of the primary particles of second raw material 62. Therefore, composite particles having a smaller particle size can be manufactured.

Note that the method of mixing the raw materials is not limited to the above example, and other methods capable of mixing, carrying, and coating can be used. Furthermore, the form of the raw material is not limited to a solid powder, and may be a slurry in which a liquid, a gas, or a solid powder is dispersed in a dispersion medium. Furthermore, composite particles can be obtained even if the raw materials are used without being mixed.

(b) In the composite particle formation step (step S2), first raw material 61 of composite raw material 60 obtained in the mixing step (step S1) is micronized by a thermal plasma method and carried on the surface of second raw material 62 to manufacture composite particles 80.

A thermal plasma device 100 illustrated in FIG. 2 is used to micronize first raw material 61 of composite raw material 60.

### <Thermal Plasma Device>

The thermal plasma device 100 includes at least reaction chamber 20 as an example of vacuum chamber, material supply unit 10, a thermal plasma generator including a plurality of electrodes 43 as illustrated in, for example, FIG. 3, and a composite particle collection unit (here, bag filter 50) as an example of a collection device that collects generated composite particle.

Reaction chamber 20 is surrounded by a grounded cylindrical reaction chamber wall. Material supply unit 10 supplies composite raw material 60 into reaction chamber 20.

The thermal plasma generator generates thermal plasma at about 2000° C. to 10,000° C. using, for example, AC power. In the thermal plasma generator, the plurality of electrodes 43 are disposed at predetermined intervals on the side portion of the central portion of reaction chamber 20 so as to penetrate from the outside to the inside and a tip of each electrode protrudes into an internal space.

Bag filter 50 is disposed closer to reaction chamber 20 than dry pump 30, and collects composite particles 80 generated in reaction chamber 20.

In such thermal plasma device 100, thermal plasma 70 is generated in reaction chamber 20, and the first raw material of composite raw material 60 supplied from material supply unit 10 is instantaneously evaporated by generated thermal plasma 70 and rapidly cooled in the gas phase, whereby composite particle 80 in which the first raw material is carried on the surface of the second raw material can be manufactured.

The micronization step (step S2) performed using the thermal plasma device further includes, for example, steps of (1) raw material introduction and vacuuming, (2) gas introduction and pressure adjustment, (3) discharge start and plasma generation, (4) raw material supply, (5) composite particle formation, and (6) discharge stop and composite particle collection.

Each of the above steps will be described below.

(1) First, raw material introduction and vacuuming are performed. Specifically, for example, composite raw material 60 obtained by mixing TiO₂ (second raw material) and CuO (first raw material) is introduced into material supply unit 10. Subsequently, reaction chamber 20, the inside of a pipe in which the composite particle collection unit (not illustrated) is disposed, and the inside of material supply unit 10 are evacuated by dry pump 30, thereby reducing the influence of retained oxygen. Note that, although not illustrated, the composite particle collection unit includes a cyclone capable of classifying particles having an arbitrary particle size or more and bag filter 50 capable of collecting desired composite particles 80.

(2) Gas introduction and pressure adjustment are performed. Specifically, gas is supplied from each of a plurality of gas supply devices A and B to material supply unit 10 and gas supply pipes 40 and 41 while adjusting the flow rate, and conductance valve 31 adjusts the pressure of reaction chamber 20 to a predetermined pressure. In the present exemplary embodiment, for example, argon gas is introduced as the discharge gas.

(3) Discharge is started to generate plasma. Specifically, a predetermined voltage is applied to the plurality of electrodes 43 of the plasma generator in FIG. 3 to discharge (arc discharge). The, the arc discharge is ignited to generate thermal plasma 70. When the current applied to each electrode is stabilized after the arc discharge is ignited, composite raw material 60 is supplied from material supply unit 10 to reaction chamber 20.

(4) A gas is supplied from each of the plurality of gas supply devices A and B to material supply unit 10, and composite raw material 60 is supplied to reaction chamber 20 together with the gas. Specifically, composite raw material 60 is sent together with the gas from material supply unit 10 to material supply pipe 42, and is introduced together with the gas from material supply pipe 42 to reaction chamber 20. As a carrier gas for supplying composite raw material 60 to reaction chamber 20, for example, argon gas is used.

Note that a plurality of gas supply pipes 40 and 41 for sending composite raw material 60 and composite particles 80 formed by discharge in a certain direction (In FIG. 2, the lower side in the longitudinal direction of the paper surface (−Z direction)) is provided around material supply pipe 42. The gas is supplied from gas supply pipes 40 and 41 in the above certain direction.

(5) Next, composite particles 80 are formed. When composite raw material 60 supplied to reaction chamber 20 together with the gas passes through a region where thermal plasma 70 is generated (hereinafter, thermal plasma 70), first raw material 61 is evaporated or vaporized (hereinafter, it is referred to as “evaporation”), and first raw material 61 is gasified. Thermal plasma 70 is controlled such that first raw material 61 is evaporated and second raw material 62 is not evaporated. The control of thermal plasma 70 can be realized by controlling at least one of a temperature of the thermal plasma, a heating time by the thermal plasma, a gas species of the thermal plasma, a pressure of the thermal plasma, a supply position, a flow rate, and a gas species of a cooling gas of the thermal plasma, a type of the composite raw material, average particle sizes of the primary particle and the secondary particle, a carried form, and a supply medium of the composite raw material. For example, it is possible to generate thermal plasma 70 at about 2500° C. to 5000° C. in which CuO as the first raw material is evaporated and TiO₂ as the second raw material is not evaporated by adjusting the pressure. Furthermore, for example, by changing a distance between the counter electrodes of the plurality of electrodes 43 of the plasma generator, the number of electrodes, a frequency of the AC power applied to the electrodes, or a timing of the AC power applied to the electrodes, an arbitrary temperature can be set in a wide range. Furthermore, the heating time can be controlled by causing a gas to flow downstream of thermal plasma 70. For example, it goes without saying that there are various combinations depending on the combination of the particle size and shape of the raw material and the temperature and treatment time of the thermal plasma depending on the plasma conditions. By controlling the thermal plasma as described above, it is possible to evaporate the first raw material and to suppress the evaporation of the second raw material to, for example, 10 wt % or less.

On the other hand, in the above temperature range, TiO₂ as second raw material 62 is melted because it is not less than the melting point and not more than the evaporation temperature. The mixed raw material of the molten particles of second raw material 62 and the gas of first raw material 61 flows in the above-described certain direction by the flow of the gas from gas supply pipes 40 and 41, and at the moment of leaving thermal plasma 70, the molten particles of second raw material 62 are rapidly cooled in the gas phase and solidified in a substantially spherical shape. Furthermore, the gas generated from first raw material 61 is also rapidly cooled and solidified in the gas phase, and carried as fine particles on the surface of second raw material 62 to generate composite particles 80. The cooling rate at this time is, for example, about 10⁴ K/sec to 10⁵ K/sec. In the case, first, an element having a high melting point is solidified, and then an element having a lower melting point is solidified. Therefore, second raw material 62 including an element having a high melting point serves as base material particles, and composite particles 80 in which fine particles generated from first raw material 61 including an element having a low melting point are carried on the surfaces of the base material particles are formed. In the case of titanium and copper, titanium oxide that is an oxide of titanium having a high melting point serves as base material particles, and composite particles in which copper element-containing particles containing copper having a lower melting point are carried on the surfaces of the base material particles are generated.

Note that second raw material 62 may be evaporated in an amount of 10 wt % or less. Even if a part of second raw material 62 evaporates, fine particles of second raw material 62 having a high melting point are first generated on the surface of the unevaporated particles of the second raw material, and then fine particles of first raw material 61 having a low melting point are generated. Therefore, the fine particles of first raw material 61 are present on the outermost surface of second raw material 62. Therefore, there is no influence on the characteristics, and there is no influence on the fluidity of generated composite particles 80 as long as the evaporation is 10 wt % or less. Moreover, since a part of second raw material 62 is evaporated and fine particles generated from first raw material 61 are carried on the surface, the surface area that can be used for the reaction can be increased, and the performance can be further improved.

Note that the mixed material of the molten particles of the second raw material and the gas of the first raw material may be naturally cooled, but is not limited thereto. For example, the terminal portion of thermal plasma 70 may be cooled by a cooling gas (not illustrated) introduced from cooling gas supply pipes 90, 91 (FIG. 2). By increasing a cooling rate by the cooling gas, it is possible to suppress the evaporation of second raw material 62 and to suppress the aggregation of the fine particles generated from the gas of first raw material 61, and it is possible to manufacture the composite particles dispersed and carried more uniformly. In FIG. 2, gas supply pipes 40, 41, material supply pipe 42, and cooling gas supply pipes 90, 91 are connected to each other for the sake of simplicity, but this does not mean that these pipes are connected at all times. The same or different gases may be selectively supplied to the respective pipes as necessary.

(6) Next, the discharge is stopped, and generated composite particles 80 are collected. Composite particles 80 generated by thermal plasma 70 are collected by bag filter 50 by a flow of gas (carrier gas and discharge gas) from gas supply pipes 40 and 41 toward the composite particle collection unit (not illustrated). As illustrated in FIG. 2, bag filter 50 is installed in front of dry pump 30 for exhaust.

When a desired amount of composite raw material 60 is treated, the discharge is stopped, and the generation of thermal plasma 70 is stopped. Then, composite particles 80 collected by bag filter 50 are taken out. At this time, composite particle 80 may be taken out, for example, under an inert gas atmosphere such as nitrogen gas. Oxidation of composite particles 80 can be suppressed by taking out the composite particles under an inert gas atmosphere.

In the present the first exemplary embodiment, an example in which TiO₂ and CuO are used as raw materials of composite particles 80 has been described, but as a raw material of TiO₂, any of anatase type TiO₂, rutile type TiO₂, brookite type TiO₂, or a mixture thereof can be used because the evaporation temperature is the same. The crystal form of the base material particles may be controlled by controlling the proportion of these raw materials. Furthermore, a Cu₂O source can be used because any of Cu and Cu compounds such as CuO, Cu, Cu₂O, and CuCl₂ or mixtures thereof can be evaporated. A proportion of Cu₂O in the copper element-containing particles may be controlled by controlling a proportion of these raw materials.

Furthermore, in the present first exemplary embodiment, an example in which a solid powder raw material is used as first raw material 61 has been described, but a liquid containing Cu can also be evaporated, and thus can be used.

Furthermore, in the first exemplary embodiment, an example in which CuO is used as the first raw material has been described, but the first raw material is not limited thereto. As the first raw material, a raw material including one type of element selected from copper, molybdenum, and silver may be used.

Furthermore, in the first exemplary embodiment, an example in which TiO₂ is used as the second raw material of composite particles 80 has been described, but the second raw material is not limited thereto. As the second raw material, a raw material including one or more types of elements selected from aluminum, titanium, zirconium, hafnium, iron, yttrium, niobium, tantalum, silicon, calcium, magnesium, tungsten, indium, tin, germanium, nickel, zinc, and molybdenum may be used.

Note that, in the method of manufacturing composite particle 80 according to the present exemplary embodiment, the thermal plasma method is used, but other methods may be used as long as fine particles having an average particle size of 300 nm or less can be manufactured by evaporating the first raw material such as CuO and rapidly cooling the first raw material. In the thermal plasma method, high frequency thermal plasma, direct current arc plasma, or alternating current arc plasma may be used, and as a method other than the thermal plasma method, a flame method using a burner, a laser ablation method, or a thermal decomposition method using a high frequency heating method or the like may be used.

Note that, although an example in which only argon gas is used as the gas has been described, the present disclosure is not limited thereto. At least one gas of the material supply gas (carrier gas), the discharge gas, and the gas (cooling gas) (not illustrated) introduced from cooling gas supply pipes 90, 91 to the terminal portion of thermal plasma 70 may be used by adding oxygen gas to inert gas such as argon gas.

Furthermore, in the case of synthesizing composite particles of TiO₂ and Cu₂O, when only an inert gas is used, a part of oxygen derived from a raw material generated by evaporation of the raw material cannot contribute to the reaction, and a part of oxygen is lost, so that metal Cu may be generated. Note that, in the mixed gas of the inert gas and the oxygen gas, the content of the oxygen gas is, for example, 0.1 vol % to 50 vol %. By adding oxygen gas to the inert gas, oxygen deficiency can be suppressed, and a ratio of Cu₂O can be increased.

Furthermore, at least one gas of the carrier gas, the discharge gas, and the cooling gas may be used by adding oxygen gas and hydrogen gas or a carbon-based reducing gas to inert gas such as argon gas. The oxidation and crystal structure of the oxide of the first and second raw materials may be controlled by the oxygen gas and/or the reducing gas. Note that, when the oxygen gas is excessively added, a proportion of CuO in Cu₂O, CuO, and Cu constituting the copper element-containing particles in the first raw material increases, and a proportion of Cu₂O decreases. Therefore, the proportion of Cu₂O can be optimized by further adding hydrogen gas or a carbon-based reducing gas. Furthermore, since Cu₂O is generated at a temperature lower than that of the oxide of the base material particles, a gas to which hydrogen gas or a carbon-based reducing gas is added may be introduced as a cooling gas from the terminal portion of thermal plasma 70. Furthermore, the cooling gas may be supplied upward (Z direction) from the bottom of reaction chamber 20 so as to be countercurrent to the thermal plasma.

Furthermore, in the above description, the oxidation and crystal structure of the oxide of the base material particles of the second raw material and/or the copper element-containing particles in the first raw material are controlled by controlling the atmosphere of the oxygen gas and/or the reducing gas, but the present disclosure is not limited thereto. For example, among the raw materials, a ratio of Cu₂O, CuO, and Cu in the copper element-containing raw material in the first raw material may be controlled to control a ratio of Cu₂O in the copper element-containing particles. This makes it possible to control the ratio of Cu₂O in the copper element-containing particles in the first raw material without using a reducing agent.

### [Composite Particle]

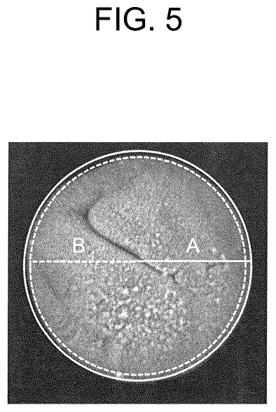

Subsequently, the composite particle according to the first exemplary embodiment will be described with reference to FIG. 5. FIG. 5 is an electron microscope image of composite particle 80 (hereinafter, composite particle of Example 1) obtained by the method of manufacturing composite particle 80 according to the first exemplary embodiment. Note that the entire width of FIG. 5 is about 10 μm.

Composite particles 80 according to the first exemplary embodiment have a shape in which Cu₂O fine particles having an average particle size of 0.5 nm or more and 300 nm or less are carried on the surfaces of spherical TiO₂ particles having an average particle size of more than 5 μm and 10 μm or less from the electron microscope image and the elemental analysis in FIG. 5. That is, in the composite particles, TiO₂ particles mainly including a rutile type are base material particles, and Cu₂O fine particles are present on the surfaces of the base material particles. The Cu₂O fine particles are present on the surfaces of the base material particles, and the surfaces of the TiO₂ particles as the base material particles is not entirely covered, so that photocatalytic activity can be obtained together with high antimicrobial or antiviral performance.

Furthermore, as illustrated in FIG. 5, the base material particle generated from second raw material 62 has a substantially spherical shape. That is, the particle is a spherical particle without a corner that is a pointed portion created by intersection of two surfaces. Furthermore, a value of the sphericity represented by a ratio (A−B)/A of a difference (A−B) between a radius B of the smallest spherical surface inscribed in the surface of the particle and a maximum value A of a distance from the center of the smallest spherical surface to the surface of the particle to the maximum value A was 0.1 or less. Although depending on the material properties, dispersibility is good as long as the sphericity is 0.3 or less. A sphericity of 0.1 or less is preferable because dispersibility is further improved. Therefore, since the composite particle according to the first exemplary embodiment has good fluidity, the dispersibility of the material is high when the composite particle is kneaded with a resin, ceramic, or metal to form a molded body, and the material can be uniformly dispersed in the molded body.

Note that an average particle size of the primary particles of each of the base material particles and the copper element-containing particles is obtained, for example, by calculating the number average of 100 particles in an electron microscope or a transmission electron image. The average particle size can be measured by, for example, a dynamic light scattering method.

Furthermore, in the present first exemplary embodiment, a mixture of TiO₂ and CuO at a weight ratio of 99.5:0.5 is used as a raw material, but the mixing ratio of composite particles 80 can also be controlled by changing the mixing ratio of TiO₂ and CuO. When the proportion of CuO is excessively less than 0.01 wt %, Cu₂O is reduced, and the antiviral property is deteriorated. Conversely, when the proportion of CuO is increased, TiO₂ is covered with Cu₂O, the antimicrobial or antiviral performance of Cu₂O becomes strong, and the photoresponsiveness is lowered, but deterioration at the time of resin mixing can be suppressed, and coloring can be suppressed more than Cu₂O alone. The proportion of CuO may be increased to 10 wt %. When the content is more than 10 wt %, photoresponsiveness may not be sufficiently obtained.

Note that, in addition, the oxide of the base material particle including the second raw material may be an oxide or a composite oxide including one or more types of elements selected from aluminum, titanium, zirconium, hafnium, iron, yttrium, niobium, tantalum, silicon, calcium, magnesium, tungsten, indium, tin, germanium, nickel, zinc, and molybdenum. In the case of the oxide or composite oxide including the elected elements, since the particles are white particles, coloring can be suppressed as compared with the case of using Cu₂O alone.

Note that, in addition, the carried particles generated from the first raw material may be copper or molybdenum element-containing particles including at least one of cuprous oxide, copper oxide, copper, molybdenum oxide, silver, or silver oxide. Since the molybdenum oxide is white particles, coloring can be suppressed as compared with the case of using Cu₂O alone.

Therefore, in a resin composition containing composite particles produced by thermal plasma, a resin molded body, or a resin sheet-shaped molded body, for example, when the mixing amount is 5 wt % or less, mixing can be performed while maintaining the color of the main component.

As a resin, composite particles are kneaded with a resin mainly including polypropylene this time, but the resin is not limited thereto. The resin may be, for example, a resin mainly composed of polyethylene, polystyrene, acryl, methacryl, polyethylene terephthalate (PET), polycarbonate, or the like.

Furthermore, in a molded body of metal or ceramic containing composite particles produced by thermal plasma, for example, when the mixing amount is 5 wt % or less, mixing can be performed while maintaining the color of the main component.

Note that the present disclosure includes appropriate combination of arbitrary exemplary embodiments and/or examples among the various exemplary embodiments and/or examples described above, and effects of the respective exemplary embodiments and/or examples can be exhibited.

### INDUSTRIAL APPLICABILITY

According to the method of manufacturing a composite particle according to the present disclosure, it is possible to easily obtain composite particles in which, using a second raw material of a selected element as a base material particle, fine particles generated from a first raw material including at least one of cuprous oxide, copper oxide, copper, molybdenum oxide, silver, or silver oxide and having an average particle size of 0.5 nm or more and 300 nm or less are present on a surface of the base material particle of the second raw material. The composite particles have high catalytic performance or antimicrobial or antiviral performance and high dispersibility. Moreover, it is possible to produce a large amount of composite particles in a short time with less contamination of impurities, and it is useful as a method of manufacturing a composite particle.

### REFERENCE MARKS IN THE DRAWINGS

10: material supply unit
20: reaction chamber
30: dry pump
31: conductance valve
40, 41: gas supply pipe
42: material supply pipe
43: electrode
50: bag filter
60: composite raw material
61: first raw material
62: second raw material
70: thermal plasma
80: composite particle
90, 91: cooling gas supply pipe

1. A method of manufacturing a composite particle, the method comprising:
a step of preparing a first raw material including an element selected from any of copper, molybdenum, and silver, and a second raw material including one or more types of elements selected from aluminum, titanium, zirconium, hafnium, iron, yttrium, niobium, tantalum, silicon, calcium, magnesium, tungsten, indium, tin, germanium, nickel, zinc, and molybdenum; and
a thermal plasma evaporation and cooling step of introducing the prepared first and second raw materials into thermal plasma to evaporate the first raw materials, and cooling the evaporated first raw materials to generate a composite particle,
wherein the composite particle includes the second raw material, and a fine particle carried on a surface of the second raw material and generated from the first raw material having an average particle size of 0.5 nm or more and 300 nm or less.

2. The method according to claim 1, wherein the thermal plasma evaporation and cooling step includes a step of controlling the thermal plasma not to evaporate more than or equal to 10 wt % of the second raw material.

3. The method according to claim 1, wherein the second raw material has a melting point higher than a melting point of the first raw material.

4. The method according to claim 1, wherein
in the step of preparing the first raw material and the second raw material, the first and second raw materials are composite raw materials, and
the composite raw material includes the second raw material that is a particle and the first raw material carried or coated on a surface of the second raw material.

5. The method according to claim 1, wherein the second raw material is a particle, and the particle of the second raw material has no corner that is a pointed portion created by intersection of two surfaces.

6. The method according to claim 1, wherein both the first raw material and the second raw material are particles, and the first raw material has an average particle size of 0.2 times or less an average particle size of the second raw material.

7. The method according to claim 1, wherein the second raw material is a secondary particle granulated from a primary particle.

8. The method according to claim 1, wherein the thermal plasma evaporation and cooling step includes a step of supplying a cooling gas to a terminal portion of the thermal plasma.

9. The method according to claim 1, wherein the thermal plasma evaporation and cooling step includes a step of controlling the thermal plasma to preferentially evaporate the first raw material by controlling at least one of a temperature of the thermal plasma, a heating time by the thermal plasma, a temperature distribution of the thermal plasma, a gas species of the thermal plasma, a pressure of the thermal plasma, a supply position, a flow rate, and a gas species of a cooling gas of the thermal plasma, a type of a composite raw material in which the first raw material is carried or coated on a surface of a particle of the second raw material, average particle sizes of a primary particle and a secondary particle, a carrying form, and a supply medium of the composite raw material.

10. A composite particle comprising:
a base material particle having an average particle size of more than 0.3 μm and 100 μm or less and including a compound containing an oxide of one type of element selected from aluminum, titanium, zirconium, hafnium, iron, yttrium, niobium, tantalum, silicon, calcium, magnesium, tungsten, indium, tin, germanium, nickel, zinc, and molybdenum; and
a fine particle having an average particle size of 0.5 nm or more and 300 nm or less, containing at least one of cuprous oxide, copper oxide, copper, molybdenum oxide, silver, or silver oxide, and being present on a surface of the base material particle.

11. A composite particle comprising:
a base material particle having an average particle size of more than 0.3 μm and 100 μm or less and including a compound containing an oxide of one type of element selected from aluminum, titanium, zirconium, hafnium, iron, yttrium, niobium, tantalum, silicon, calcium, magnesium, tungsten, indium, tin, germanium, nickel, zinc, and molybdenum; and
a fine particle having an average particle size of 0.5 nm or more and 300 nm or less, including a compound containing an oxide of one type of element selected from aluminum, titanium, zirconium, hafnium, iron, yttrium, niobium, tantalum, silicon, calcium, magnesium, tungsten, indium, tin, germanium, nickel, and zinc and including at least two of cuprous oxide, copper oxide, copper, molybdenum oxide, silver, or silver oxide, and being present on a surface of the base material particle.

12. The composite particle according to claim 10, wherein the base material particle has a spherical shape satisfying Formula (1) without a corner that is a pointed portion created by intersection of two surfaces:
$$( A - B ) \leq 0.3 \times A \qquad Formula ( 1 )$$
where B is a radius of a minimum spherical surface inscribed in a surface of the base material particle, and
A is a maximum value of a distance from a center of the minimum spherical surface to the surface of the base material particle.

13. A resin composition comprising:
a resin; and
the composite particle according to claim 10 in the resin.

14. A resin molded body comprising:
a resin; and
the composite particle according to claim 10 in the resin.

15. A metal and ceramic molded body comprising:
a resin; and
the composite particles according to claim 10 in the resin.
